(19) United States
(12) Patent Application Publication
(10) Pub. No.: US 2005/0075354 A1
(43) Pub. Date: Apr. 7, 2005

(54) Complexes of E-2-Methoxy-N(3-{4-[3-methyl-4-(6-methyl-pyridin-3-yloxy)-phenylamino]-quinzazolin-6-YL}-allyl)-acetamide, their method of production, and use

(75) Inventors: Zheng Jane Li, Quarker Hill, CT (US); Jason A. Leonard, Groton, CT (US); Andrew V. Trask, Stonington, CT (US); John C. Kath, Waterford, CT (US); Daniel T. Richter, Groton, CT (US); Carlton B. Thompson, Pawcatuck, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/738,972

(22) Filed: Dec. 17, 2003

Related U.S. Application Data
(60) Provisional application No. 60/434,700, filed on Dec. 19, 2002.

(51) Int. Cl. A61K 31/517; C07D 43/02

(52) U.S. Cl. 514/266.21; 544/284

(57) ABSTRACT

The invention relates to complexes of E-2-Methoxy-N-(3-{4-[3-methyl-4-(6-methyl-pyridin-3-yloxy)-phenylamino]-quinazolin-6-yl}-allyl)-acetamide having the following formula I: 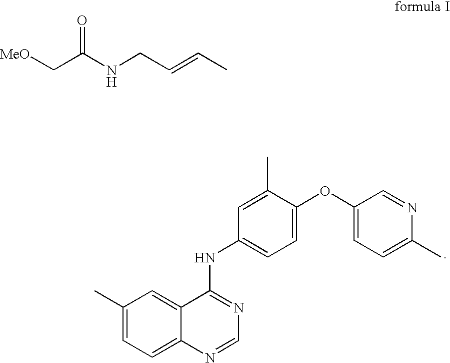 The invention also relates to pharmaceutical compositions containing the complexes of formula I. The invention further relates to methods of treating hyperproliferative diseases, such as cancers, in mammals, especially humans by administering the above complexes and to methods of preparing the above complexes.

This application claims priority from U.S. Provisional Application Ser. No. 60/434,700 Filed Dec. 19, 2003.

### BACKGROUND OF THE INVENTION

This invention relates to complexes of E-2-Methoxy-N-(3-{4-[3-methyl-4-(6-methyl-pyridin-3-yloxy)-phenylamino]-quinazolin-6-yl}-allyl)-acetamide having the formula I:

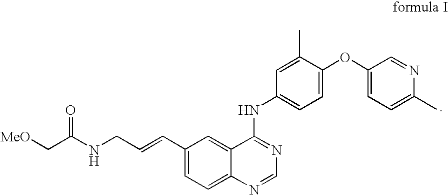

Formula I in its free base form is described in International Publication No. WO01/98277 published Dec. 27, 2001, the disclosure of which is hereby incorporated herein by reference in its entirety. The foregoing application is assigned in common with the present application. The free base of formula I is useful in the treatment of hyperproliferative diseases, such as cancers.

Succinate and malonate salt forms, including the sesquisuccinate and di-malonate salt forms of E-2-Methoxy-N-(3-{4-[3-methyl-4-(6-methyl-pyridin-3-yloxy)-phenylamino]-quinazolin-6-yl}-allyl)-acetamide were disclosed in U.S. Provisional Patent Application Ser. No. 60/340,885, filed Dec. 12, 2001.

The present invention further relates to particular complexes of E-2-Methoxy-N-(3-{4-[3-methyl-4-(6-methyl-pyridin-3-yloxy)-phenylamino]-quinazolin-6-yl}-allyl)-acetamide. The invention also relates to pharmaceutical compositions containing these complexes. The complexes of the present invention are useful in the treatment of hyperproliferative diseases, such as cancers, in mammals, especially humans. The invention also relates to methods of administering these complexes to treat hyperproliferative diseases.

### SUMMARY OF THE INVENTION

The present invention relates to complexes of E-2-Methoxy-N-(3-{4-[3-methyl-4-(6-methyl-pyridin-3-yloxy)-phenylamino]-quinazolin-6-yl}-allyl)-acetamide having the following formula I:

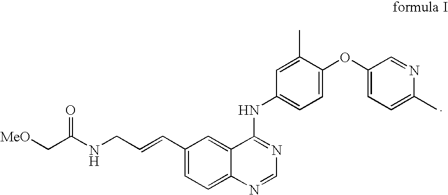

Examples of such complexes include the maleate (including the dimaleate), hydrochloride (including monohydrochloride), succinate (including the sesquisuccinate and monosuccinate), malonate (including the dimalonate), phosphate (including monophosphate), fumarate (including monofumarate), hemiedisylate, tartrates (including both racemic and optically active forms), camsylate (including both racemic and optically active forms), besylate, esylate, nitrate, and citraconate (including dicitraconate) complexes of formula I.

The present invention also relates to a complex formed by contacting E-2-Methoxy-N-(3-{4-[3-methyl-4-(6-methyl-pyridin-3-yloxy)-phenylamino]-quinazolin-6-yl}-allyl)-acetamide with an acid or an reactive equivalent of said acid, wherein said acid is at least one member selected from the group consisting of maleic acid, hydrochloric acid, and phosphoric acid.

The present invention also relates to a method for the inhibition of abnormal cell growth in a mammal comprising administering to said mammal an amount of the above mentioned complex that is effective in inhibiting abnormal cell growth.

The present invention also relates to a method for treating a mammal having a disease (such as cancer) characterized by an overexpression of erbB2, comprising administering to the mammal the above-mentioned complex in an amount that is effective in treating the disease.

The present invention also relates to a method for inducing cell death comprising exposing a cell which overexpresses erbB2 to an effective amount of the above-mentioned compolex.

The present invention also relates to a pharmaceutical composition comprising an amount of an above-mentioned complex effective to treat a hyperproliferative disorder in a mammal, and a pharmaceutically acceptable carrier.

### BRIEF DESCRIPTION OF THE SEVERAL VIEWS OF THE DRAWING(S)

FIG. 1 is a X-ray powder diffraction spectrum of the E-2-Methoxy-N-(3-{4-[3-methyl-4-(6-methyl-pyridin-3-yloxy)-phenylamino]-quinazolin-6-yl}-allyl)-acetamide monohydrochloride which was prepared and isolated according to Example 5.

FIG. 2 is a X-ray powder diffraction spectrum of the E-2-Methoxy-N-(3-{4-[3-methyl-4-(6-methyl-pyridin-3-yloxy)-phenylamino]-quinazolin-6-yl}-allyl)-acetamide dimaleate which was prepared and isolated according to Example 6.

FIG. 3 is a X-ray powder diffraction spectrum of the E-2-Methoxy-N-(3-{4-[3-methyl-4-(6-methyl-pyridin-3-yloxy)-phenylamino]-quinazolin-6-yl}-allyl)-acetamide monophosphate (monohydrate) described in Example 7.

### DETAILED DESCRIPTION OF THE INVENTION

The present invention relates to complexes of E-2-Methoxy-N-(3-{4-[3-methyl-4-(6-methyl-pyridin-3-yloxy)-phenylamino]-quinazolin-6-yl}-allyl)-acetamide having the following formula I:

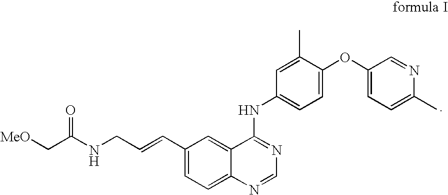

Examples of such complexes include the maleate (including the dimaleate), hydrochloride (including monohydrochloride), succinate (including the sesquisuccinate and monosuccinate), malonate (including the dimalonate), phosphate (including monophosphate), fumarate (including monofumarate), hemiedisylate, tartrates (including both racemic and optically active forms), camsylate (including both racemic and optically active forms), besylate, esylate, nitrate, and citraconate (including dicitraconate) complexes of formula I.

In one preferred embodiment the invention relates to hydrochloride, maleate and phosphate complexes of E-2-Methoxy-N-(3-{4-[3-methyl-4-(6-methyl-pyridin-3-yloxy)-phenylamino]-quinazolin-6-yl}-allyl)-acetamide.

In one particularly preferred embodiment, the hydrochloride complex is a monohydrochloride complex, the maleate complex is a dimaleate complex and the phosphate complex is a monophosphate complex.

In a preferred embodiment, the dimaleate, the monohydrochloride and the monophosphate complexes are substantially salts.

In one embodiment, the presently disclosed monohydrochloride, monophosphate and dimaleate complexes of E-2-Methoxy-N-(3-{4-[3-methyl-4-(6-methyl-pyridin-3-yloxy)-phenylamino]-quinazolin-6-yl}-allyl)-acetamide are amorphous and in one embodiment (preferred), crystalline, i.e., substantially free of amorphous material (i.e., at least 90% crystalline, and in one embodiment, at least 95% crystalline, and in one embodiment at least 99% crystalline). Such crystalline materials can provide more reproducible dosing results. They have optimum properties of aqueous solubility, chemical and physical stability and bioavailability for pharmaceutical compositions. Generally they have relatively higher solubility and bioavailability than the starting E-2-Methoxy-N-(3-{4-[3-methyl-4-(6-methyl-pyridin-3-yloxy)-phenylamino]-quinazolin-6-yl}-allyl)-acetamide from which they are prepared. The stability of these materials may also alleviate potential problems associated with weight changes of active ingredients during manufacture of capsules or tablets.

In one embodiment, the hydrochloride, dimaleate, and monophosphate are crystalline materials that exhibit an X-ray powder diffraction spectrum having characteristic peaks expressed in degrees (2θ) and relative intensities (RI) as disclosed in Examples 3, 4 and 5 respectively.

The dimaleate, monophosphate and monohydrochloride complexes of E-2-Methoxy-N-(3-{4-[3-methyl-4-(6-methyl-pyridin-3-yloxy)-phenylamino]-quinazolin-6-yl}-allyl)-acetamide are chemically stable and are non-hygroscopic, which may alleviate potential problems associated with weight changes of the active ingredient during the manufacture of capsules or tablets.

The present invention also relates to a complex formed by contacting E-2-Methoxy-N-(3-{4-[3-methyl-4-(6-methyl-pyridin-3-yloxy)-phenylamino]-quinazolin-6-yl}-allyl)-acetamide with an acid or a reactive equivalent of said acid, wherein said acid is at least one member selected from the group consisting of maleic acid, hydrochloric acid, and phosphoric acid.

In one embodiment, wherein the acid is maleic acid, the complex is E-2-Methoxy-N-(3-{4-[3-methyl-4-(6-methyl-pyridin-3-yloxy)-phenylamino]-quinazolin-6-yl}-allyl)-acetamide maleate and preferably E-2-Methoxy-N-(3-{4-[3-methyl-4-(6-methyl-pyridin-3-yloxy)-phenylamino]-quinazolin-6-yl}-allyl)-acetamide dimaleate.

In one embodiment, wherein the acid is hydrochloric acid, the complex is E-2-Methoxy-N-(3-{4-[3-methyl-4-(6-methyl-pyridin-3-yloxy)-phenylamino]-quinazolin-6-yl}-allyl)-acetamide hydrochloride and preferably E-2-Methoxy-N-(3-{4-[3-methyl-4-(6-methyl-pyridin-3-yloxy)-phenylamino]-quinazolin-6-yl}-allyl)-acetamide monohydrochloride.

In one embodiment, wherein the acid is phosphoric acid, the complex is E-2-Methoxy-N-(3-{4-[3-methyl-4-(6-methyl-pyridin-3-yloxy)-phenylamino]-quinazolin-6-yl}-allyl)-acetamide phosphate and preferably E-2-Methoxy-N-(3-{4-[3-methyl-4-(6-methyl-pyridin-3-yloxy)-phenylamino]-quinazolin-6-yl}-allyl)-acetamide monophosphate.

The present invention also relates to a method for the inhibition of abnormal cell growth in a mammal which comprises administering to said mammal an amount of the aforementioned complexes of E-2-Methoxy-N-(3-{4-[3-methyl-4-(6-methyl-pyridin-3-yloxy)-phenylamino]-quinazolin-6-yl}-allyl)-acetamide, that is effective in inhibiting abnormal cell growth.

In one embodiment the abnormal cell growth treated is cancer.

In one embodiment of the present the cancer is selected is selected from lung cancer, non small cell lung (NSCL) cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), colorectal cancer (CRC), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In a preferred embodiment of the present invention, cancer is selected from breast cancer, colon cancer, ovarian cancer, non small cell lung (NSCL) cancer, colorectal cancer (CRC), prostate cancer, bladder cancer, renal cancer, gastric cancer, endometrial cancer, head and neck cancer, and esophageal cancer.

In a more preferred embodiment of the present invention, the cancer is selected from renal cell carcinoma, gastric cancer, colon cancer, breast cancer, and ovarian cancer.

In a more preferred embodiment, the said cancer is selected from colon cancer, breast cancer or ovarian cancer.

Another embodiment of the present invention relates to method for the inhibition of abnormal cell growth in a mammal which comprises administering to said mammal an amount of the complex of E-2-Methoxy-N-(3-{4-[3-methyl-4-(6-methyl-pyridin-3-yloxy)-phenylamino]-quinazolin-6-yl}-allyl)-acetamide that is effective in inhibiting abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

In a preferred embodiment, the complex is combined with a cytotoxic.

In one preferred embodiment of the present invention the cytotoxic is Taxol® (paclitaxel).

The present invention further relates to a method for the inhibition of abnormal cell growth in a mammal which comprises administering to said mammal an amount of the complex of E-2-Methoxy-N-(3-{4-[3-methyl-4-(6-methyl-pyridin-3-yloxy)-phenylamino]-quinazolin-6-yl}-allyl)-acetamide, that is effective in inhibiting abnormal cell growth in combination with a compound selected from the group consisting of Cyclophosphamide, 5-Fluorouracil, Floxuridine, Gemcitabine, Vinblastine, Vincristine, Daunorubicin, Doxorubicin, Epirubicin, Tamoxifen, Methylprednisolone, Cisplatin, Carboplatin, CPT-11, gemcitabine, paclitaxel, and docetaxel.

In one preferred embodiment, the above compound is selected from the group consisting Tamoxifen, Cisplatin, Carboplatin, paclitaxel and docetaxel.

The invention further relates to a pharmaceutical composition for the inhibition of abnormal cell growth in a mammal comprising an amount of the complex of E-2-Methoxy-N-(3-{4-[3-methyl-4-(6-methyl-pyridin-3-yloxy)-phenylamino]-quinazolin-6-yl}-allyl)-acetamide, that is effective in inhibiting abnormal cell growth, and a pharmaceutically acceptable carrier.

In one embodiment, the pharmaceutical composition further comprises an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method for treating a mammal having a disease characterized by an overexpression of erbB2, comprising administering to the mammal, the complex of E-2-Methoxy-N-(3-{4-[3-methyl-4-(6-methyl-pyridin-3-yloxy)-phenylamino]-quinazolin-6-yl}-allyl)-acetamide in an amount that is effective in treating said disease characterized by the overexpression of erbB2.

In a preferred embodiment, the disease is cancer.

The invention also relates to a method inducing cell death comprising exposing a cell which overexpresses erbB2 to an effective amount of the complex of E-2-Methoxy-N-(3-{4-[3-methyl-4-(6-methyl-pyridin-3-yloxy)-phenylamino]-quinazolin-6-yl}-allyl)-acetamide. In one embodiment the cell is a cancer cell in a mammal, preferably a human.

The present invention relates to a method inducing cell death comprising exposing a cell which overexpresses erbB2 to an effective amount of the complex of E-2-Methoxy-N-(3-{4-[3-methyl-4-(6-methyl-pyridin-3-yloxy)-phenylamino]-quinazolin-6-yl}-allyl)-acetamide, and said method further comprises exposing the cell to a growth inhibitory agent.

In one preferred embodiment the cell is exposed to a chemotherapeutic agent or radiation.

The invention further relates to a method of treating cancer in a human, wherein the cancer expresses the erbB2 receptor, comprising administering to the human a therapeutically effective amount of the complex of E-2-Methoxy-N-(3-{4-[3-methyl-4-(6-methyl-pyridin-3-yloxy)-phenylamino]-quinazolin-6-yl}-allyl)-acetamide that has reduced affinity for the erbB1 receptor. In one preferred embodiment of the present invention the cancer is not characterized by overexpression of erbB1 receptor. In another preferred embodiment the cancer is characterized by overexpression of the erbB1 and erbB2 receptor.

This invention also relates to a method for the treatment of a disorder associated with angiogenesis in a mammal, including a human, comprising administering to said mammal the complex of E-2-Methoxy-N-(3-{4-[3-methyl-4-(6-methyl-pyridin-3-yloxy)-phenylamino]-quinazolin-6-yl}-allyl)-acetamide, or solvate or prodrug thereof, that is effective in treating said disorder. Such disorders include cancerous tumors such as melanoma; ocular disorders such as age-related macular degeneration, presumed ocular histoplasmosis syndrome, and retinal neovascularization from proliferative diabetic retinopathy; rheumatoid arthritis; bone loss disorders such as osteoporosis, Paget's disease, humoral hypercalcemia of malignancy, hypercalcemia from tumors metastatic to bone, and osteoporosis induced by glucocorticoid treatment; coronary restenosis; and certain microbial infections including those associated with microbial pathogens selected from adenovirus, hantaviruses, Borrelia burgdorferi, Yersinia spp., Bordetella pertussis, and group A Streptococcus.

“Complex”, as used herein, unless otherwise indicated, refers to an acid-base pair that has a defined stoichiometry and contains ionized, unionized and/or partially charged base and acid species, wherein the extent of proton transfer from acid (proton donor) to the base (proton acceptor) can vary in proportions from none, partial, to all. All complexes can be termed with the suffix “ate” or “ide” to represent a complex of a specific acid whose name ends in “ic”. For example, a complex of a basic compound with succinic acid wherein the mole ratio of succinic acid to the basic compound is 1.5 is named as a “sesquisuccinate” of the basic compound. One of ordinary skill in the art will appreciate that the above definition of “complex” includes salt wherein the extent of proton transfer from the acid to the base is substantially in full proportion (i.e., complete proton transfer).

“Substantially salt” as used herein, refers to a complex, wherein the extent of proton transfer from the acid to the base is at least about 90%, and in one embodiment, at least about 95%, and in one embodiment, at least about 99%.

“Reactive equivalent of a material” as used herein, refers to any compound or chemical composition other than the material itself, which reacts like the material itself under the reaction conditions. Thus reactive equivalents of carboxylic acids will include acid-producing derivatives such as anhydrides, acyl halides, and mixtures thereof unless specifically stated otherwise. One of ordinary skill in the art that will recognize that the phrase “synthon” is a synonym for “reactive equivalent”.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) expressing an activated Ras oncogene; (2) tumor cells in which the Ras protein is activated as a result of oncogenic mutation in another gene; (3) benign and malignant cells of other proliferative diseases in which aberrant Ras activation occurs; and (4) any tumors that proliferate by virtue of farnesyl protein transferase.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “a compound that has reduced affinity for the erbB1 receptor”, as used herein, unless otherwise indicated, means wherein the compound is an erbB2 inhibitor and has a range of selectivities for erbB2 receptor over the erbB1 receptor between 50-1500, i.e., the compound is from 50 to 1500 times more selective for the erbB2 receptor over the erbB1 receptor. In a preferred embodiment the erbB2 inhibitor has a range of selectivities for erbB2 over erbB1 between 60-1200. In a more preferred embodiment the erbB2 inhibitor has a range of selectivities for erbB2 over erbB1 between 80-1000. In an even more preferred embodiment the erbB2 inhibitor has a range of selectivities for erbB2 over erbB1 between 90-500. In a most preferred embodiment the erbB2 inhibitor has a range of selectivities for erbB2 over erbB1 between 100-300. In the most preferred embodiment the erbB2 inhibitor has a range of selectivities for erbB2 over erbB1 between 110-200. The selectivity of the erbB2 inhibitor over the erbB1 inhibitor is measured using the whole cell (intact) assay described below.

Each of the documents referred to herein is incorporated by reference in its entirety, for all purposes. Except in the Examples, or where otherwise explicitly indicated, all numerical quantities in this description specifying amounts of materials, degree of crystallinity, degree of proton transfer from acid the base in the description of “complex” hereinabove, reaction and process conditions (such temperature, time, pressure), and the like are to be understood to be modified by the word “about”.

The in vitro activity of the complexes of E-2-Methoxy-N-(3-{4-[3-methyl-4-(6-methyl-pyridin-3-yloxy)-phenylamino]-quinazolin-6-yl}-allyl)-acetamide may be determined by the following procedure.

The in vitro activity of the complexes of E-2-Methoxy-N-(3-{4-[3-methyl-4-(6-methyl-pyridin-3-yloxy)-phenylamino]-quinazolin-6-yl}-allyl)-acetamide as erbB kinase inhibitors in intact cells may be determined by the following procedure. Cells, for example 3T3 cells transfected with human EGFR (Cohen et al. J. Virology 67:5303, 1993) or with chimeric EGFR/erbB2 kinase (EGFR extracellular/erbB2 intracellular, Fazioli et al. Mol. Cell. Biol. 11: 2040, 1991) are plated in 96-well plates at 12,000 cells per well in 100 μl medium (Dulbecco's Minimum Essential Medium (DMEM) with 5% fetal calf serum, 1% pen/streptomycin, 1% L-glutamine) and incubated at 37° C., 5% CO₂. Test compounds are solubilized in DMSO at a concentration of 10 mM, and tested at final concentrations of 0, 0.3 μM, 1 μM, 0.3 μM, 0.1 μM and 10 μM in the medium. The cells are incubated at 37° C. for 2 h. EGF (40 ng/ml final) is added to each well and cells incubate at room temperature for 15 min followed by aspiration of medium, then 100 μl/well cold fixative (50% ethanol/50% acetone containing 200 micromolar sodium orthovanadate) is added. The plate is incubated for 30 min at room temperature followed by washing with wash buffer (0.5% Tween 20 in phosphate buffered saline). Blocking buffer (3% bovine serum albumin, 0.05% Tween 20, 200 μM sodium orthovanadate in phosphate buffered saline, 100 μl/well) is added followed by incubation for 2 hours at room temperature followed by two washes with wash buffer. PY54 monoclonal anti-phosphotyrosine antibody directly conjugated to horseradish peroxidase (50 μl/well, 1 μg/ml in blocking buffer) or blocked conjugate (1 μg/ml with 1 mM phosphotyrosine in blocking buffer, to check specificity) is added and the plates incubated for 2 hours at room temperature. The plate wells are then washed 4 times with wash buffer. The colorimetric signal is developed by addition of TMB Microwell Peroxidase Substrate (Kirkegaard and Perry, Gaithersburg, Md.), 50 μl per well, and stopped by the addition of 0.09 M sulfuric acid, 50 μl per well. Absorbance at 450 nM represents phosphotyrosine content of proteins. The increase in signal in EGF-treated cells over control (non-EGF treated) represents the activity of the EGFR or EGFR/chimera respectively. The potency of an inhibitor is determined by measurement of the concentration of compound needed to inhibit the increase in phosphotyrosine by 50% (IC₅₀) in each cell line. The selectivity of the compounds for erbB2 vs. EGFR is determined by comparison of the IC₅₀ for the EGFR transfectant vs. that for the erbB2/EGFR chimera transfectant. Thus, for example, a compound with an IC₅₀ of 100 nM for the EGFR transfectant and 10 nM for the erbB2/EGFR chimera transfectant is considered 10-fold selective for erbB2 kinase.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumor substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

Where HPLC chromatography is referred to in the preparations and examples below, the general conditions used, unless otherwise indicated, are as follows. The column used is a ZORBAX™ RXC18 column (manufactured by Hewlett Packard) of 150 mm distance and 4.6 mm interior diameter. The samples are run on a Hewlett Packard-1100 system. A gradient solvent method is used running 100 percent ammonium acetate/acetic acid buffer (0.2 M) to 100 percent acetonitrile over 10 minutes. The system then proceeds on a wash cycle with 100 percent acetonitrile for 1.5 minutes and then 100 percent buffer solution for 3 minutes. The flow rate over this period is a constant 3 mL/minute.

In the following examples and preparations, “Et” means ethyl, “AC” means acetyl, “Me” means methyl, “ETOAC” or “ETOAc” means ethyl acetate, “THF” means tetrahydrofuran, and “Bu” means butyl.

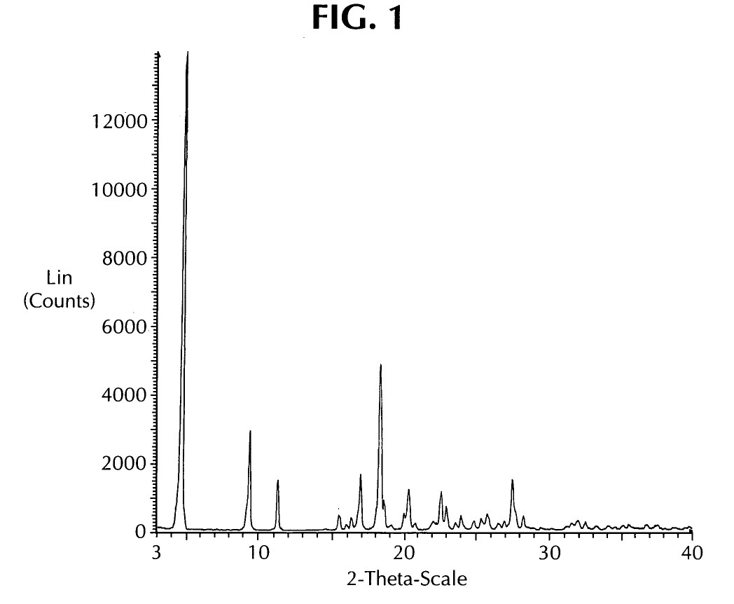

The spectrums in FIGS. 1-3 were recorded using a Bruker¹ D5000 diffractometer equipped with copper radiation, fixed slits (1.0,1.0,0.6 mm), and a Kevex solid state detector. Data was collected from 3.0 to 40.0 degrees in two theta using a step size of 0.04 degrees and a step time of 1.0 seconds.

The experimental conditions under which the powder X-ray diffraction was conducted are as follows: Cu anode; wavelength 1: 1.54056 angstrom; wavelength 2: 1.54439 angstrom (Relative Intensity: 0.500); range # 1-coupled: 3.000 to 40.000; step size: 0.040; step time: 1.00; smoothing width: 0.300; and threshold: 1.0.

For single crystal X-ray analysis, data collection was done through a Bruker CCD diffractometer. Cu anode: wavelength 1.54178 angstrom; room temeprature;

The following details pertain to data analysis: Atomic scattering factors were taken the International Tables for X-ray Crystallography (Vol. IV, pp. 55, 99, 149 Birmingham: Kynoch Press, 1974). All crystallographic calculations were facilitated by the SHELXTL system G. M. Sheldrick, SHELXTL, User Manual, Nicholet Instrument Co., 1981). A trial structure was obtained by direct methods.

Calculation of PXRD pattern from single crystal data: To compare the results between a single crystal and a powder sample, a powder X-ray pattern based on the single crystal structural data can be calculated. The calculation can be done using SHELXTL Plus computer program, Reference Manual by Siemens Analytical X-ray Instrument, Chapter 10, p. 179-181, 1990. The single crystal structural data provide the cell dimensions, space group and atomic positions of a crystal form. These parameters are used as the basis to calculate a perfect powder pattern of that crystal form. Comparing the calculated PXRD pattern and the experimental pattern will confirm whether a powder sample corresponds to an assigned single crystal structure. This procedure has been performed on the crystal forms of azithromycin, A, D, F, G and J. The results are displayed in the overlaid powder X-ray diffraction patterns with the lower pattern as the calculated from single crystal data and the upper one as a representative experimental pattern. A match between the two patterns indicates the agreement between powder sample and the corresponding single crystal structure.

### EXAMPLE 1

### Free Base of E-2-Methoxy-N-(3-{4-[3-methyl-4-(6-methyl-pyridin-3-yloxy)-phenylamino]-quinazolin-6-yl}-allyl)-acetamide

The free base of E-2-Methoxy-N-(3-{4-[3-methyl-4-(6-methyl-pyridin-3-yloxy)-phenylamino]-quinazolin-6-yl}-allyl)-acetamide is prepared according Example 182 (LMRS: 470.1, HPLC RT:5.05) using procedure G described in PCT Publication WO 01/98277, the disclosure of which is hereby incorporated herein by reference in its entirety. Procedure G WO 01/98277, is shown below.

### Method G: Synthesis of E-N-(3-{4-[3-Chloro-4-(6-methyl-pyridin-3-yloxy)-phenylamino]-quinazolin-6-yl}-allyl)-acetamide (7)

E-(3-{4-[3-chloro-4-(6-methyl-pyridin-3-yloxy)-phenylamino]-quinazolin-6-yl}-allyl)-carbamic acid tert-butyl ester: To a solution of 7.53 mL of a 65% weight toluene solution of sodium bis(2-methoxyethoxy)aluminum hydride (Red-Al, 24.2 mmol) in 90 mL of tetrahydrofuran at 0° C. was added 5.0 g of (3-{4-[3-chloro-4-(6-methyl-pyridin-3-yloxy)-phenylamino]-quinazolin-6-yl}-prop-2-ynyl)-carbamic acid tert-butyl ester as a solid. The reaction was stirred at 0° C. for 2 hours, quenched with 10% aqueous potassium carbonate and extracted with ethyl acetate. The combined organics were dried and evaporated. The crude material was purified on 115 g of silica gel, eluting with 80% ethyl acetate/hexanes to afford 4.42 g of E-(3-{4-[3-chloro-4-(6-methyl-pyridin-3-yloxy)-phenylamino]-quinazolin-6-yl}-allyl)-carbamic acid tert-butyl ester. ¹H NMR (CDCl₃): δ 8.66 (s, 1), 8.24 (m, 1), 8.03 (m, 2), 7.77-7.65 (m, 3), 7.13 (m, 2), 6.97 (d, J=8.7 Hz, 1), 6.54 (d, 1), 6.35 (m, 1), 4.9 (m, 1), 3.90 (m, 2), 2.52 (s, 3), 1.46 (s, 9).

E-[6-(3-amino-propenyl)-quinazolin-4-yl]-[3-chloro-4-(6-methyl-pyridin-3-yloxy)-phenyl]-amine. To a solution of 4.42 g of E-(3-{4-[3-chloro-4-(6-methyl-pyridin-3-yloxy)-phenylamino]-quinazolin-6-yl}-allyl)-carbamic acid tert-butyl ester in 21 mL of tetrahydrofuran was added 21 mL of 2 N hydrochloric acid. The mixture was heated at 60° C. for 3 hours, cooled to room temperature and basified with 10% aqueous potassium carbonate. Methylene chloride was added to the aqueous mixture and a solid precipitated. The solid was filtered and dried to yield 2.98 g of E-[6-(3-amino-propenyl)-quinazolin-4-yl]-[3-chloro-4-(6-methyl-pyridin-3-yloxy)-phenyl]-amine. ¹H NMR (d₆ DMSO): δ 8.62 (s, 1), 8.53 (m, 1), 8.26 (m, 2), 7.99 (m, 1), 7.89 (m, 1), 7.77 (m, 1), 7.30 (m, 3), 6.67 (m, 2), 3.44 (m, 2), 2.47 (s, 3).

E-N-(3-{4-[3-Chloro-4-(6-methyl-pyridin-3-yloxy)-phenylamino]-quinazolin-6-yl}-allyl)-acetamide. A mixture of 14.4 μL (0.25 mmol) of acetic acid and 40.3 mg (0.33 mmol) of dicyclohexylcarbodiimide in 2 mL of methylene chloride were stirred for 10 minutes and treated with 100.3 mg of E-[6-(3-amino-propenyl)-quinazolin-4-yl]-[3-chloro-4-(6-methyl-pyridin-3-yloxy)-phenyl]-amine. The reaction was allowed to stir at room temperature overnight. The precipitate which formed was filtered and chromatographed on silica gel, eluting with 6-10% methanol/chloroform to afford 106 mg of the title compound; m.p. 254-256° C.; ¹H NMR (d₆ DMSO): δ 9.88 (s, 1), 8.58 (s, 1), 8.48 (m, 1), 8.20 (m, 3), 7.95 (m, 1), 7.83 (m, 1), 7.71(d, J=8.7 Hz, 1), 7.24 (m, 2), 7.19 (d, J=8.7 Hz, 1), 6.61 (d, J=16.2 Hz, 1), 6.48 (m, 1), 3.90 (m, 2).

### EXAMPLE 2

### Free Base of E-2-Methoxy-N-(3-{4-[3-methyl-4-(6-methyl-pyridin-3-yloxy)-phenylamino]-quinazolin-6-yl}-allyl)-acetamide

The following procedure for prepareing the free base of E-2-Methoxy-N-(3-{4-[3-methyl-4-(6-methyl-pyridin-3-yloxy)-phenylamino]-quinazolin-6-yl}-allyl)-acetamide is disclosed in U.S. Provisional Application Ser. No. 60/334,647, filed Nov. 30, 2001:

### Synthesis of 6-Iodo-[3-methyl-4-(6-methyl-pyridine-3-yloxy)-phenylamino]-quinazoline

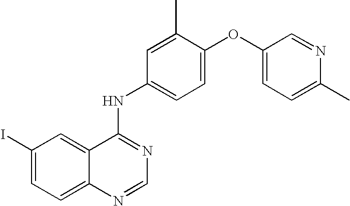

A 3 neck round bottom flask was fitted with a mechanical stirrer and kept under N₂. The flask was charged with the 6-iodo-4-chloroquinazoline (10.0 g, 34.43 mol) and dry THF (35 ml). Thereafter, 3-methyl-4-(6-methyl-pyridine-3-yloxy)-phenylamine (7.38 g, 34.43 mmol) and dry THF (45 ml) were added and the yellow suspension was heated to reflux. After 15 minutes most of the reactants went into solution and a fine yellow suspension was obtained. After 25 min, the internal temperature of the reaction mixture was 56° C., and precipitation of the desired product started. Heating was continued for a further 2 hours and the reaction mixture was allowed to cool to room temperature while remaining in the oil bath. Yellow crystals were collected by filtration, washed with cold (0° C.) THF (1×10 ml) and dried at 50° C., p<200 mbar. The title compound was obtained as light yellow crystals (15.75 g, 98%). Rf=0.45 (EtOAc/MeOH=9/1). ¹H NMR (CDCl₃, 300 MHz): δ=11.40 (br, s, 1H, NH), 9.29 (d, J=Hz, 1H, H-2), 8.91 (s, 1H, H-2″), 8.36-8.32 (m, 2H, H-7, H-8), 7.74-7.73 (m, 2H, H-4″, H-5), 7.62 (dd, J₁=8.7 Hz, J₂=2.6 Hz, 1H, H-5″) 7.49-7.46 (m, 2H, H-6′, H-5), 7.06 (d, J=8.7 Hz, 1H H-2′), 2.54 (s, 3H, CH₃), 2.26 (s, 3H, CH₃). ¹³C NMR (CDCl₃+D₆-DMSO, 75 MHz): δ=159.51, 153.63, 153.17, 152.82, 152.70, 145.26, 141.37, 138.01, 134.75, 134.65, 131.05, 129.10, 128.74, 126.77, 124.86, 124.43, 120.41, 116.98, 94.89, 23.54, 17.67.

The title compound had a t_{R} (min) of 12.13 under the following RP-HPLC conditions: Symmetry Shield RP18, 75×4.6 mm; Flow 1.0 mL/min; 205/210/220/245 nm; Temp. 25° C.; Injection Volume: 10 μL of a ca. 0.5% solution in ACN/H₂O 9/1; Eluent: B: ACN, C: 0.01 mmol NH₄OAc in H₂O pH=6.0; and Gradient: 0 min: B=30%, C=70%; and 20 min: B=85%, C=15%.

### Synthesis of 2-Methoxy-acetic Acid Propargylamide

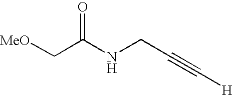

A solution of methoxy acetyl chloride (12.5 ml, 0.137 mol, 1.2 equiv.) in dry CH₂Cl₂ (45 ml) kept under N₂ was cooled to −40° C. A solution of propargylamine (7.98 ml, 0.125 mol, 1.0 equiv.) in dry CH₂Cl₂ (40 ml) was added over 45 minutes keeping the temperature less than-25° C. After 15 minutes triethylamine (17.4 ml, 0.125 mol, 1.0 equiv.) was added over 45 minutes keeping the temperature less than −25° C. The reaction mixture was warmed to room temperature. TLC after 3 hours showed conversion complete. The reaction mixture was quenched with H₂O (50 ml) and the organic phase was washed with half-saturated NaCl solution, filtered through cotton wool and concentrated at a temperature of 40° C. and pressure of greater than 650 mbar. The crude compound was purified by short path distillation (boiling point of 49° C. and p of 0.09 mbar). The title compound was obtained as a colorless liquid (7.84 g, 50%) which crystallized upon standing.

R_{f}=0.36 (heptane/EtOAc=7/3).

¹H NMR (CDCl₃, 300 MHz): δ=6.72 (br, s, 1H, N—H), 4.09 (dd, J₁=5.5 Hz, J₂=2.6 Hz, 2H, CH₂—NH), 3.92 (s, 2H, CH₂—OMe), 3.43 (s, 3H, OCH₃), 2.24 (t, J=2.6 Hz, 1H, alkyne CH).

¹³C-NMR (CDCl₃, 75 MHz): δ=169.14 (C═O), 79.11 (C-2′), 71.63 (C-2), 71.41 (C-3′), 59.04 (OCH₃), 28.26 (C-1′).

Gas chromatography was used to determine the t_{R} (min) of 6.42 under the conditions shown in the table below.

| | |
|---|---|
| Column | DB-5 (30 m × 0.32 mm, 0.25 μm film thickness) |
| Injector | Split, initial Temp. 250° C. |
| Split ratio | 60.243:1 |
| Split flow | 108.3 ml/min, gas type: hydrogen |
| Oven | 60° C., 1 min, 10° C./min, 290° C., 10 min |
| Inject-Temp | 250° C. |
| Detector (FID) | Detector Temp. 250° C. |
| Detector flow | H₂: 40.0 ml/min, air: 450 ml/min |
| Makeup flow | N₂: 45.0 ml/min |

### Preparation of 6-(N-Methoxyacetyl-3-amino-propen-1-yl)-4-[3-methyl-4-(6-methyl-pyridine-3-yloxy)-phenylamino]-quinazoline (Which is E-2-Methoxy-N-(3-{4-[3-methyl-4-(6-methyl-pyridin-3-yloxy)-phenylamino]-quinazolin-6-yl}-allyl)-acetamide) Using Suzuki Coupling Reaction

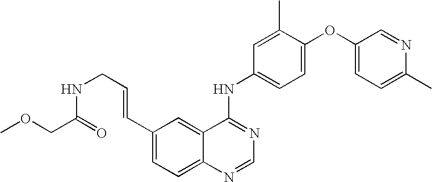

2-methyl-2-butene (0.59 ml, 5.60 mmol, 2.8 equiv.) was added over 1 hour to a cold (0-5° C.) solution of BH₃*THF complex (1.0 M sol, 3.0 ml, 3.0 mmol, 1.5 equiv.) kept under N₂. The reaction mixture was stirred at this temperature for 30 minutes followed by the addition of 2-Methoxy-acetic acid propargylamide (255 mg, 2 mmol, 1.0 equiv.) dissolved in dry THF (1 ml) over 15 minutes. The ice-bath was removed and the reaction mixture was warmed to room temperature over 20 minutes. The reaction mixture was then heated at 35° C. for 1 hour. K₂CO₃ (0.55 g, 4 mmol, 2.0 equiv.) dissolved in degassed H₂O (1.2 ml) was added over 30 minutes to the reaction mixture. During the addition of the first half gas evolution was observed which seized during further addition. 6-Iodo-[3-methyl-4-(6-methyl-pyridine-3-yloxy)-phenylamino]-quinazoline (1.41 g, 3 mmol, 1.5 equiv.) was added in three portions giving a yellow suspension. PPh₃ (21 mg, 0.08 mmol, 4 mol %) and Pd(OAc)₂ (4.5 mg, 0.02 mmol, 1 mol %) were added each in one portion and the reaction mixture was heated to reflux (65-68° C.). After about 30 minutes a yellow solution was obtained and the reaction was monitored by HPLC assay. After 18 hours the reaction mixture was cooled to room temperature followed by the addition of half-saturated NaCl solution (10 ml) and EtOAc (10 ml). The organic phase was separated, washed with H₂O (5 ml) and concentrated at 50° C. and a pressure of less than 200 mbar. Purification by plug filtration, SiO₂, EtOAc/MeOH=9/1. The title compound was obtained as light yellow crystals (0.55 g, 59%). R_{f}=0.16 (EtOAc/MeOH=9/1). ¹H-NMR (CDCl₃, 250 MHz): δ=8.71 (s, 1H, H-2), 8.25 (d, J=1.7 Hz, 1H, H-8), 7.90(s, 1H, H-7), 7.82 (s, 1H, NH), 7.79 (s, 1H, H-5), 7.66 (d, J=2.5 Hz, 1H, H-4″), 7.54 (dd, J₁=8.7 Hz, J₂=2.6 Hz, 1H, H-5″), 7.15-7.07 (m, 2H, H-5′, H-6′), 6.91 (d, J=8.7 Hz, 1H, H-2′), 6.83 (bt, 1H, NH), 6.65 (d, J=15.9 Hz, 1H, H-9), 6.34 and 6.29 (dt, J₁=15.9 Hz, J₂=6.1 Hz, 1H, H-10), 4.14 (dt, J=6.1 Hz, 2H, CH₂OMe), 3.97 (s, 2H, CH₂NH), 3.45 (s, 3H, OCH₃), 2.53 (s, 3H, CH₃), 2.29 (s, 3H, CH₃). ¹³C-NMR (CDCl₃, 75 MHz): δ=169.76 (C═O), 157.90, 154.93, 152.367, 152.23, 150.90, 149.74, 139.34, 134.73, 134.63, 131.16, 130.77, 130.36, 128.85, 129.98, 125.47, 124.66, 123.65, 121.32, 119.51, 119.13, 115.39, 71.96, 59.26, 40.84, 23.57, 16.41.

Using reverse phase high performance liquid chromatography t_{R} (min) was found to be 6.02 for the title compound under the conditions shown in the following table.

| | |
|---|---|
| Symmetry Shield RP18 | 75 × 4.6 mm |
| Flow | 1.0 mL/min |
| Wavelength | 205/210/220/245 nm |
| Temp. | 25° C. |
| Injection Volume | 10 μL of a ca. 0.5% solution in ACN/H₂O 9/1 |
| Eluent B | ACN |
| Eluent C | 0.01 mmol NH₄OAc in H₂O pH = 6.0 |
| Gradient 0 min | B = 30%, C = 70% |
| Gradient 20 min | B = 85%, C = 15% |

### EXAMPLE 3

### Free Base of E-2-Methoxy-N-(3-{4-[3-methyl-4-(6-methyl-pyridin-3-yloxy)-phenylamino]-quinazolin-6-yl}-allyl)-acetamide

The free base form of E-2-Methoxy-N-(3-{4-[3-methyl-4-(6-methyl-pyridin-3-yloxy)-phenylamino]-quinazolin-6-yl}-allyl)-acetamide can also be prepared by neutralization of the corresponding dimesylate salt.

The dimesylate salt is prepared as follows:

To 67.33 grams of the free base form E-2-Methoxy-N-(3-{4-[3-methyl-4-(6-methyl-pyridin-3-yloxy)-phenylamino]-quinazolin-6-yl}-allyl)-acetamide (prepared according to Example 1 above) in 400 mL of EtOH and 100 ml of CH₂Cl₂ at room temp was added dropwise a soln of 19.17 mL (2.05) equivs of methanesulfonic acid (CH₃SO₃H) in 100 ml of acetonitrile. The mixture was slurried at room temperature for 15 minutes then the methylene chloride (˜100 ml) was removed. An additional 600 mL of acetonitrile was added to complete crystallization and the mixture slurried for 2 hours. The crystals were filtered under a nitrogen atmosphere and washed with 100 ml of acetonitrile. The dimesylate salt (94.48 grams) was produced in 99% yield.

The dimesylate salt produced according to the method of the preceding paragraph (90 g) was dissolved in water (˜550 mL). Chloroform was added (˜500 mL) to the solution followed by 1N NaOH until a white suspension/precipitate was observed (pH ˜13-14). The addition of chloroform before NaOH reduced gumming as the precipitate formed. The mixture was transferred to a separatory funnel (2 L) and the free base was extracted with three portions of chloroform (˜300 mL). The extracts were combined (˜1.3 L), washed with water (˜500 mL), dried with anhydrous magnesium sulfate, and then filtered. The chloroform filtrate was concentrated in vacuo to provide a yellow amorphous solid/oil. This material was reslurried in ethyl acetate overnight resulting in a white solid. This material was then filtered, washed with cold ethyl acetate, and then dried in a vacuum oven at 45° C. to yield a white crystalline solid (˜59 g). The free base was characterized by polarizing light microscopy (PLM), powder X-ray diffraction (PXRD), and differential scanning calorimetry (DSC). It is in the form of a needle, and displays three endothermic events by DSC (DSC melting points: 125° C., 160° C., and 167° C.)

### EXAMPLE 4

### Synthesis of E-2-Methoxy-N-(3{4-[3-methyl-4-(6-methyl-pyridin-3-yloxy)-phenylamino]-quinazolin-6-yl}-allyl)-acetamide Monohydrochloride

A solution of E-2-Methoxy-N-(3-{4-[3-methyl-4-(6-methyl-pyridin-3-yloxy)-phenylamino]-quinazolin-6-yl}-allyl)-acetamide in isopropyl alcohol was prepared by dissolving 500 mg E-2-Methoxy-N-(3-{4-[3-methyl-4-(6-methyl-pyridin-3-yl oxy)-phenylamino]-quinazolin-6-yl}-allyl)-acetamide, using the procedure of Example 1, 2 or 3 in 50 mL isopropanol with stirring. The solution was heated to 75° C. Then, concentrated hydrochloric acid (1.1 equivalents; 115 mg) was diluted with 6 mL of isopropanol. The diluted HCl solution was added drop-wise to the hot free base solution with stirring. After complete addition, heat was removed from solution and a microcrystalline precipitate emerged upon cooling to ambient temperature over about three hours. The thick yellow slurry was stirred one day and filtered. The fine yellow powder was collected by vacuum filtration and dried under vacuum. The yield was approximately 79%.

The hydrochloride salt was determined to be an anhydrous monohydrochloride salt by combustion analysis. The compound exhibited a melting endotherm at 222° C. by DSC at a heating rate of 5° C./min. Its PXRD pattern is shown in FIG. 1. Characteristic X-ray powder diffraction peaks (2-theta (±0.1°) [% relative intensity]): 4.6 [100], 9.3 [20.9], 11.4 [10.6], 15.6 [3.4], 16.4 [2.8], 17.1 [11.8], 18.4 [34.8], 18.8 [5.9], 20.1 [3.8], 20.4 [8.6], 22.6 [8.2], 23.0 [5.1], 24.0 [3.3], 25.4 [2.7], 25.8 [3.7], 27.5 [10.7], and 28.3 [3.2].

### EXAMPLE 5

### Synthesis of E-2-Methoxy-N-(3-{4-[3-methyl-4-(6-methyl-pyridin-3-yloxy)-phenylamino]-quinazolin-6-yl}-allyl)-acetamide Dimaleate

A solution of maleic acid was prepared by dissolving 2.2 equivalents of maleic acid in 7:3 (v/v) CHCl₃/EtOH. E-2-Methoxy-N-(3-{4-[3-methyl-4-(6-methyl-pyridin-3-yloxy)-phenylamino]-quinazolin-6-yl}-allyl)-acetamide (prepared according to Example 1, 2 or 3 above) was dissolved in 70:30 CHCl₃/EtOH (v/v), and added drop-wise to the maleic acid solution with stirring. After about 2 days, white crystalline powder precipitated.

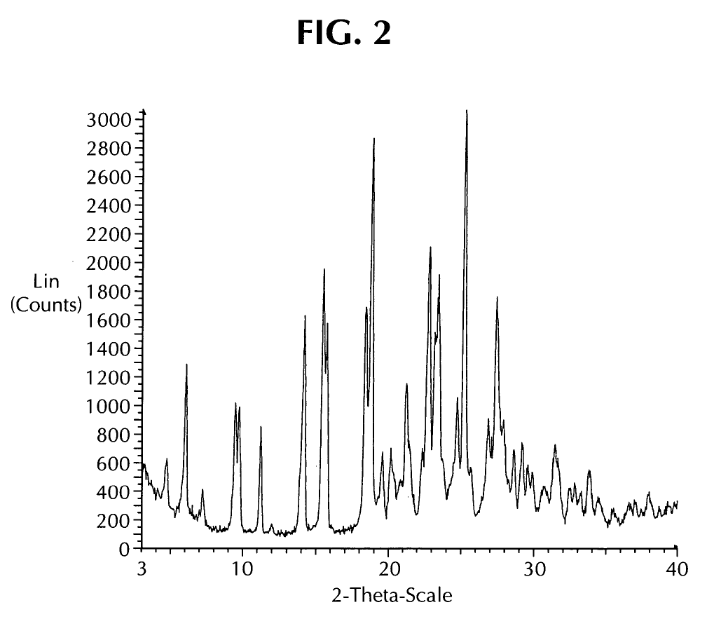

By polarized light microscopy, the dimaleate crystals had a needle habit with strong birefringence. On hot-state polarizing light microscope (PLM), the crystals melted/decomposed at ˜170° C. The DSC thermogram showed an endotherm at ˜170° C. immediately followed by an exotherm. The endotherm and exotherm correspond with the melt/decomposition events seen by hot-stage PLM. Hygroscopicity: 0.6% (by weight) at 90% relative humidity. The PXRD is shown in FIG. 2. Characteristic X-ray powder diffraction peaks (2-theta (+0.1°), [% relative intensity]): 4.6 [20.4], 6.0 [41.9], 7.2 [13.1], 9.4 [33], 9.7 [32], 11.2 [27.7], 12.0 [5.2], 14.1 [20], 14.2 [53], 15.5[63.7], 15.7 [51.2], 18.4 [55], 18.7 [93.4], 19.3 [5], 19.6 [21.9], 20.2 [22.9], 20.4 [16.2], 20.8 [15.5], 21.2 [37.6], 22.4 [22.7], 22.8 [68.7], 23.2 [49.2], 23.4 [62.5], 23.8 [18.8], 24.5 [8.7], 24.8 [34.3], 25.2 [100], 25.7 [18.4], 26.4 [11.5], 26.9 [29.5], 27.1 [10.8], 27.4 [57.4], 27.7 [14.3], 27.9 [29.2], 28.4 [9.4], 28.6 [22.4], 29.2 [24], 29.6 [18.9], 29.9 [17.2], 30.7 [13.9], and 31.4 [23.7]. Calculated X-ray diffraction peaks (from single crystal) (2-theta (±0.1°), [% relative intensity]): 4.7 [21], 6.0 [34.5], 7.2 [18.3], 9.5 [32.3], 9.7 [25.9], 11.3 [32], 12.1 [1.7] 14.0 [20.3], 14.2 [37.8], 15.6 [37.5], 15.8 [42.1], 18.4 [59.7], 18.8 [100], 19.3 [15.9], 19.7 [22.9], 20.2 [22.9], 20.5 [16.5], 20.8 [18.6], 21.3 [58.8], 22.4 [29.5], 22.8 [75.9], 23.3 [48.3], 23.5 [55.7], 23.9 [18.4], 24.6 [18.1], 24.8 [30.3], 25.3 [94.5], 25.8 [14.6], 26.5 [5.1], 26.9 [22.4], 27.1 [20.8], 27.5 [48.4], 27.8 [22.8], 28.0 [23.2], 28.3 [10], 28.7 [15.4], 29.3 [16.3], 29.7 [8.6], 29.9 [8.7], 30.8 [8.3], and 31.5 [11.6].

Single crystal x-ray data are shown in Table 3.

TABLE 3
Single crystal x-ray data for E-2-Methoxy-N-(3-{4-[3-
methyl-4-(6-methyl-pyridin-3-yloxy)-phenylamino]-
quinazolin-6-yl}-allyl)-acetamide dimaleate

| | Dimaleate |
|---|---|
| Empirical formula | C₂₇H₂₉N₅O₃²⁺.2(C₄H₃O₄⁻) |
| Formula weight | 701.68 |
| Crystal size (mm) | 0.03 × 0.04 × 0.20 |
| Space group | P-1 triclinic |
| Unit cell dimensions | a = 4.7763 (4) Å |
| | b = 19.0308 (14) Å |
| | c = 19.1520 (14) Å |
| | α = 100.4° |
| | β = 90.2° |
| | γ = 95.3° |
| Z (per formular) | 2 |
| Density (g/cm³) | 1.367 |
| R | 0.0648 |

### EXAMPLE 6

### Synthesis of E-2-Methoxy-N-(3{4-[3-methyl-4-(6-methyl-pyridin-3-yloxy)-phenylamino]-quinazolin-6-yl}-allyl)-acetamide Monophosphate

The monophosphate was prepared as the following. A free base solution was made by dissolving 5.022 grams of E-2-Methoxy-N-(3-{4-[3-methyl-4-(6-methyl-pyridin-3-yloxy)-phenylamino]-quinazolin-6-yl}-allyl)-acetamide, made according to the method of Example 1, 2 or 3 in 300 mL of ethanol and heated to 35° C. to a clear solution. One equivalent mole of phosphoric acid (87%, 0.77 mL) was diluted with 20 mL of ethanol. The acid solution was added to the free base ethanol solution drop-wise with stirring and heat (−45 to 55° C.). Yellow precipitate appeared immediately. The slurry became thick with time, 50 mL of ethyl acetate was added and the slurry was let cool to ambient temperature. The yellow crystalline powder was collected by filtration and dried under vacuum for 2 hours. The yield of the monophosphate product was about 84%. The monophosphate may contain 1-3% water.

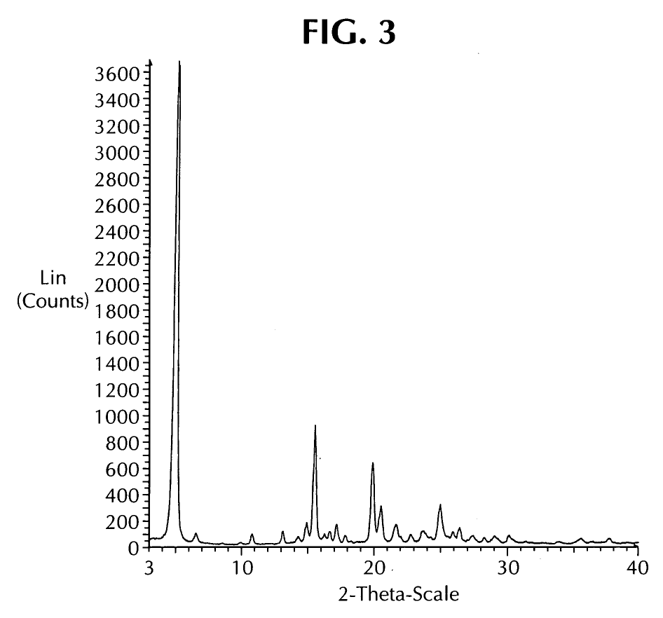

A powder x-ray diffraction pattern of the monophosphate (monohydrate) is shown in FIG. 3. Characteristic X-ray powder diffraction peaks of the monophosphate (monohydrate) (2-theta (±0.1°), [% relative intensity]): 4.9 [100], 6.5 [2.7], 10.8 [2.6], 13.1 [3], 14.3 [2], 14.9 [4.8], 15.5 [25.1], 16.3 [2.5], 16.7 [2.9], 17.2 [4.5], 17.9 [2.1], 19.9 [17.3], 20.6 [8.2], 21.7 [4.5], 22.1 [2], 22.8 [2.4], 23.7 [3.1], 24.3 [1.9], 25.0 [8.7], 26.0 [3], 26.5 [3.9], 27.5 [2.2], 28.3 [1.8], 29.1 [2.1], 30.1 [2.2], 35.5 [1.6], and 37.7 [1.6].

### EXAMPLE 7

### Synthesis of E-2-Methoxy-N-(3-{4-[3-methyl-4-(6-methyl-pyridin-3-yloxy)-phenylamino]-quinazolin-6-yl}-allyl)-acetamide Dicitraconate

A THF free base solution was prepared by dissolving 104 mg of E-2-Methoxy-N-(3-{4-[3-methyl-4-(6-methyl-pyridin-3-yloxy)-phenylamino]-quinazolin-6-yl}-allyl)-acetamide, prepared according to the method of Example 1, 2 or 3 in 5 mL of THF with stirring to a clear solution. The citraconic acid solution was prepared by dissolving 64 mg of citraconic acid (approximately 2.2 equivalents) in 1 mL of THF. The citraconic acid solution was added to the free base solution dropwise with stirring. Upon completion of the addition, no precipitate was noted. The solvent volume was reduced under a nitrogen jet, and then allowed to stir while capped. After approximately 15 minutes, trace precipitation occurred. After one hour, the solution turned into thick slurry and the slurry was allowed to stir overnight. The precipitate was then isolated using a 0.45 μm Nylon-66 membrane filter by vacuum filtration. The solids produced were rinsed with several milliliters of THF, and allowed to dry under nitrogen. The yield was approximately 62%.

Based on combustion analysis, the product was E-2-Methoxy-N-(3-{4-[3-methyl-4-(6-methyl-pyridin-3-yloxy)-phenylamino]-quinazolin-6-yl}-allyl)-acetamide dicitraconate.

### EXAMPLE 8

### Synthesis of E-2-Methoxy-N-(3-{4-[3-methyl-4-(6-methyl-pyridin-3-yloxy)-phenylamino]-quinazolin-6-yl}-allyl)-acetamide Monomalate

E-2-Methoxy-N-(3-{4-[3-methyl-4-(6-methyl-pyridin-3-yloxy)-phenylamino]-quinazolin-6-yl}-allyl)-acetamide (1 gram), prepared according to the method of either Example 1, 2 or 3 was dissolved in 25 mL hot THF. Malic acid of (571 mg; 2 molar equivalents of the free base) was added to the free base solution. The mixture was stirred overnight, during which time solids precipitated. With addition of 25 mL additional THF, the slurry was stirred an additional day and the solids were collected by vacuum filtration to yield the monomalate complex as the product.

The material was indicated to be crystalline by powder X-ray diffraction.

### EXAMPLE 9

### Synthesis of E-2-Methoxy-N-(3-{4-[3-methyl-4-(6-methyl-pyridin-3-yloxy)-phenylamino]-quinazolin-6-yl}-allyl)-acetamide Monofumarate

E-2-Methoxy-N-(3-{4-[3-methyl-4-(6-methyl-pyridin-3-yloxy)-phenylamino]-quinazolin-6-yl}-allyl)-acetamide (2 grams), prepared according to the method of Example 1, 2 or 3 was dissolved in a refluxing 16:1 (v/v) mixture of ethyl acetate (160 mL)/dichloromethane (10 mL). A solution of fumaric acid was prepared by dissolving 2 equivalents (1 gram) of fumaric acid in hot ethanol (12 mL). This acid solution was added hot to the refluxing free base solution. The resulting mixture was stirred and refluxed for approximately ten minutes, and then cooled to room temperature. Hexane (−100 mL) was added until the reaction mixture turned cloudy. The mixture was then ultrasonicated until crystals were noted. The reaction mixture was heated to approximately 70° C. and stirred overnight to produce a slurry. The solids were then collected via cold filtration to give the product.

The fumarate was a monofumarate hemipentahydrate (2.5H₂O) as determined by elemental analysis.

### EXAMPLE 10

### Synthesis of E-2-Methoxy-N-(3-{4-[3-methyl-4-(6-methyl-pyridin-3-yloxy)-phenylamino]-quinazolin-6-yl}-allyl)-acetamide Hemiedisylate

The E-2-Methoxy-N-(3-{4-[3-methyl-4-(6-methyl-pyridin-3-yloxy)-phenylamino]-quinazolin-6-yl}-allyl)-acetamide edisylate complex was synthesized by dissolving 0.5 equivalents of 1,2 ethanedisulfonic acid in 80:20 methyl ethyl ketone (MEK)/methanol (MeOH) (v/v). The E-2-Methoxy-N-(3-{4-[3-methyl-4-(6-methyl-pyridin-3-yloxy)-phenylamino]-quinazolin-6-yl}-allyl)-acetamide free base, prepared according to the method of Example 1, 2 or 3 was dissolved in approximately 60:40 MEK/MeOH (v/v), and added dropwise to the 1,2-ethanedisulfonic acid solution with stirring. Initially, an oil was formed which later crystallized into solid powders.

The material was determined to be an anhydrous hemiedisylate by elemental analysis.

### EXAMPLE 11

### Synthesis of E-2-Methoxy-N-(3-{4-[3-methyl-4-(6-methyl-pyridin-3-yloxy)-phenylamino]-quinazolin-6-yl}-allyl)-acetamide Tartrate

Several racemic E-2-Methoxy-N-(3-{4-[3-methyl-4-(6-methyl-pyridin-3-yloxy)-phenylamino]-quinazolin-6-yl}-allyl)-acetamide tartrates were produced. The synthesis of the monotartrate hemihydrate and hemitartrate hemihydrate were produced started with the production of amorphous material. This material was synthesized by dissolving 3 grams E-2-Methoxy-N-(3-{4-[3-methyl-4-(6-methyl-pyridin-3-yloxy)-phenylamino]-quinazolin-6-yl}-allyl)-acetamide free base in 20:3 (v/v) ethanol (EtOH)/dichloromethane (˜50 mL). A solution of D,L-tartaric acid was prepared by dissolving 2 grams of D,L-tartaric acid in 10 mL water. The two solutions were combined and stirred at room temperature for ˜30 minutes. The solvent was reduced to yield the amorphous material.

### EXAMPLE 12

### Synthesis of E-2-Methoxy-N-(3-{4-[3-methyl-4-(6-methyl-pyridin-3-yloxy)-phenylamino]-quinazolin-6-yl}-allyl)-acetamide Camsylates

Both the racemic and (+)-10-camphorsulfonic acid complexes of E-2-Methoxy-N-(3-{4-[3-methyl-4-(6-methyl-pyridin-3-yloxy)-phenylamino]-quinazolin-6-yl}-allyl)-acetamide were synthesized.

The (+)-10-camphorsulfonic acid complex was synthesized by dissolving 2 grams of E-2-Methoxy-N-(3-{4-[3-methyl-4-(6-methyl-pyridin-3-yloxy)-phenylamino]-quinazolin-6-yl}-allyl)-acetamide free base, prepared according to the method of Example 1, 2 or 3 in 5:2 (v/v) EtOH/Dichloromethane. The (+)-10-camphorsulfonic acid solution was produced by dissolving 1 gram of (+)-10-camphorsulfonic acid in 5 mL EtOH. The acid solution was added to the free base solution at room temperature with stirring. The reaction mixture was stirred for twenty minutes at room temperature, and the solvent volume was then reduced to yield a crude solid. A portion of the crude solid produced was dissolved in hot EtOAc. Hexanes were added until cloudy, and then the mixture was cooled to room temperature. The solution was ultrasonicated until a precipitate was noted, and then allowed to slurry at room temperature overnight. The material was isolated by filtration to yield a yellow solid.

The balance of the crude solid mentioned above was dissolved in hot EtOAc (75 mL). The solution was cooled and the seed crystals of the aforementioned yellow solid were added. The reaction mixture was then heated to ˜75° C. and slurried overnight. The mixture was cooled to RT, filtered and rinsed with EtOAc to produce (+)-of E-2-Methoxy-N-(3-{4-[3-methyl-4-(6-methyl-pyridin-3-yloxy)-phenylamino]-quinazolin-6-yl}-allyl)-acetamide camsylate.

The racemic camsylate complex was synthesized by dissolving 1 gram of E-2-Methoxy-N-(3-{4-[3-methyl-4-(6-methyl-pyridin-3-yloxy)-phenylamino]-quinazolin-6-yl}-allyl)-acetamide free base, prepared according to the method of either Example 1 or 2 in refluxing EtOAc. The acid solution was produced by dissolving 1 gram of (±)-10-camphorsulfonic acid in 15 mL of EtOAc. The acid solution was added to the refluxing free base solution. The solution was allowed to reflux overnight, and then isolated by filtration. The solids were then washed with EtOAc and dried to yield racemic E-2-Methoxy-N-(3-{4-[3-methyl-4-(6-methyl-pyridin-3-yloxy)-phenylamino]-quinazolin-6-yl}-allyl)-acetamide camsylate. Both the racemic and the (+)-camsylate samples were hygroscopic to the point of deliquescence.

### EXAMPLE 13

### Synthesis of E-2-M ethoxy-N-(3-{4-[3-methyl-4-(6-methyl-pyridin-3-yloxy)-phenylamino]-quinazolin-6-yl}-allyl)-acetamide Monobesylate

The E-2-Methoxy-N-(3-{4-[3-methyl-4-(6-methyl-pyridin-3-yloxy)-phenylamino]-quinazolin-6-yl}-allyl)-acetamide monobesylate was prepared as the following. The E-2-Methoxy-N-(3-{4-[3-methyl-4-(6-methyl-pyridin-3-yloxy)-phenylamino]-quinazolin-6-yl}-allyl)-acetamide free base, prepared according to the method of either Example 1, 2 or 3 was dissolved 500 mg in THF. Benzenesulfonic acid (168 mg, 1 molar equivalent) was added to the free base solution. Diethyl ether was then added dropwise to the solution until cloudiness was observed. After overnight stirring, the precipitate oiled out on the sides of the flask. The oily material was scraped free and allowed to stir for an additional day. Crystalline material was collected after two days.

The monobesylate had a melting onset at 135° C. by DSC, and a peak m.p. of 137° C. The material was evaluated for hygroscopicity in relative humidity chambers. After 16 hours in the 75% RH chamber there was no significant water sorption. The 94% RH chamber caused a 6.7% weight increase after the same time period, and deliquescence was observed after 16 hours in a 100% relative humidity chamber.

### EXAMPLE 14

### Synthesis of E-2-Methoxy-N-(3-{4-[3-methyl-4-(6-methyl-pyridin-3-yloxy)-phenylamino]-quinazolin-6-yl}-allyl)-acetamide Diesylate

E-2-Methoxy-N-(3-{4-[3-methyl-4-(6-methyl-pyridin-3-yloxy)-phenylamino]-quinazolin-6-yl}-allyl)-acetamide free base, prepared according to the method of either Example 1, 2 or 3 (3.00 grams) was dissolved in 40 mL ethanol and 6 mL methylene chloride. 2.05 molar equivalents of ethanesulfonic acid, dissolved in 10 mL ethanol, was added to the solution of free base. The solution was concentrated and taken up in a minimal volume of ethanol, then ethyl acetate was added as an nonsolvent until precipitation occurred. The slurry was stirred at ambient temperature over 48 hours and isolated as E-2-Methoxy-N-(3-{4-[3-methyl-4-(6-methyl-pyridin-3-yloxy)-phenylamino]-quinazolin-6-yl}-allyl)-acetamide diesylate.

The diesylate complex was crystalline by PXRD. DSC showed a clean melting onset at 146° C. and a peak at 149.5° C. Hygroscopicity: 45% (by weight) at 90% relative humidity.

### EXAMPLE 15

### Synthesis of E-2-Methoxy-N-(3{4-[3-methyl-4-(6-methyl-pyridin-3-yloxy)-phenylamino]-quinazolin-6-yl}-allyl)-acetamide Dinitrate

E-2-Methoxy-N-(3-{4-[3-methyl-4-(6-methyl-pyridin-3-yloxy)-phenylamino]-quinazolin-6-yl}-allyl)-acetamide free base (100 mg), prepared according to the method of either Example 1, 2 or 3 was dissolved in THF and 2 molar equivalents nitric acid was added. A light yellow solid precipitated and was isolated as the product.

The sample of E-2-Methoxy-N-(3-{4-[3-methyl-4-(6-methyl-pyridin-3-yloxy)-phenylamino]-quinazolin-6-yl}-allyl)-acetamide dinitrate was found to be crystalline by PXRD. Its DSC thermogram exhibited a sharp exotherm at the onset temperature of 148° C., and had a peak temperature of 151° C. Hygroscopicity: ˜7% (by weight) at 90% relative humidity.

While the invention has been explained in relation to its preferred embodiments, it is to be understood that various modifications thereof will become apparent to those skilled in the art upon reading the specification. Therefore, it is to be understood that the invention disclosed herein is intended to cover such modifications as fall within the scope of the appended claims.

1. A complex selected from E-2-Methoxy-N-(3-{4-[3-methyl-4-(6-methyl-pyridin-3-yloxy)-phenylamino]-quinazolin-6-yl}-allyl)-acetamide hydrochloride, E-2-Methoxy-N-(3-{4-[3-methyl-4-(6-methyl-pyridin-3-yloxy)-phenylamino]-quinazolin-6-yl}-allyl)-acetamide maleate, or E-2-Methoxy-N-(3-{4-[3-methyl-4-(6-methyl-pyridin-3-yloxy)-phenylamino]-quinazolin-6-yl}-allyl)-acetamide phosphate.

2. The complex of claim 1, wherein said complex is crystalline.

3. The complex of claim 1, wherein said complex is amorphous.

4. The complex of claim 1, wherein said complex is a dimaleate.

5. The complex of claim 4, wherein the dimaleate is substantially a salt.

6. The complex of claim 4, wherein the dimaleate is a crystalline material.

7. The complex of claim 6, wherein the dimaleate exhibits an X-ray powder diffraction spectrum having characteristic peaks expressed in degrees (20) at approximately:
2θ
6.0
25.2
27.1
27.7
31.4

8. The complex of claim 6, wherein the dimaleate exhibits an X-ray powder diffraction spectrum having characteristic peaks expressed in degrees (20) at approximately:

| 2θ | 2θ | 2θ |
|---|---|---|
| 4.6 | 6.0 | 7.2 |
| 9.4 | 9.7 | 11.2 |
| 12.0 | 14.1 | 4.6 |
| 6.0 | 7.2 | 24.8 |
| 25.2 | 25.7 | 26.4 |
| 26.9 | 27.1 | 27.4 |
| 27.7 | 27.9 | 28.4 |
| 28.6 | 29.2 | 29.6 |
| 29.9 | 30.7 | 31.4 |

9. The complex of claim 1, wherein said complex is a monohydrochloride.

10. The complex of claim 9, wherein the monohydrochloride is substantially a salt.

11. The complex of claim 9, wherein the monohydrochloride is a crystalline material.

12. The complex of claim 11, wherein the monohydrochloride exhibits an X-ray powder diffraction spectrum having characteristic peaks expressed in degrees (2θ) at approximately:
2θ
4.6
9.3
17.1
18.4
27.5

13. The complex of claim 11, wherein the monohydrochloride exhibits an X-ray powder diffraction spectrum having characteristic peaks expressed in degrees (28) at approximately:

| 2θ | 2θ | 2θ |
|---|---|---|
| 4.6 | 9.3 | 11.4 |
| 15.6 | 16.4 | 17.1 |
| 18.4 | 18.8 | 20.1 |
| 20.4 | 22.6 | 23.0 |
| 24.0 | 25.4 | 25.8 |
| 27.5 | 28.3 | |

14. The complex of claim 1, wherein said complex is a monophosphate.

15. The complex of claim 14, wherein the monophosphate is substantially a salt.

16. The complex of claim 14, wherein the monophosphate is a crystalline material.

17. The complex of claim 16, wherein the monophosphate exhibits an X-ray powder diffraction spectrum having characteristic peaks expressed in degrees (2θ) at approximately:
2θ
4.9
15.5
19.9
20.6
25.0

18. The complex of claim 16, wherein the monophosphate exhibits an X-ray powder diffraction spectrum having characteristic peaks expressed in degrees (2θ) at approximately:

| 2θ | 2θ | 2θ |
|---|---|---|
| 4.9 | 17.2 | 25.0 |
| 6.5 | 17.9 | 26.0 |
| 10.8 | 19.9 | 26.5 |
| 13.1 | 20.6 | 27.5 |
| 14.3 | 21.7 | 28.3 |
| 14.9 | 22.1 | 29.1 |
| 15.5 | 22.8 | 30.1 |
| 16.3 | 23.7 | 35.5 |
| 16.7 | 24.3 | 37.7 |

19. A method for the inhibition of abnormal cell growth in a mammal comprising administering to said mammal an amount of a compound of claim 1 that is effective in inhibiting abnormal cell growth.

20. A method according to claim 19, wherein said abnormal cell growth is cancer.

21. The method according to claim 20, wherein said cancer is selected from lung cancer, non small cell lung (NSCL) cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), colorectal cancer (CRC), primary CNS lymphoma, spinal axis tumors, brain stem glioma, pituitary adenoma, or a combination of one or more of the foregoing cancers.

22. The method according to claim 21, wherein said cancer is selected from breast cancer, colon cancer, ovarian cancer, non small cell lung (NSCL) cancer, colorectal cancer (CRC), prostate cancer, bladder cancer, renal cancer, gastric cancer, endometrial cancer, head and neck cancer, and esophagel cancer.

23. The method according to claim 22, wherein said cancer is selected from renal cancer, gastric cancer, colon cancer, breast cancer, and ovarian cancer.

24. The method according to claim 23, wherein said cancer is selected from colon cancer, breast cancer and ovarian cancer.

25. The method according to claim 24, wherein said cancer is breast cancer.

26. The method according to claim 24, wherein said cancer is ovarian cancer.

27. The method according to claim 24, wherein said cancer is colon cancer.

28. A method for the inhibition of abnormal cell growth in a mammal which comprises administering to said mammal an amount of a compound of claim 1 that is effective in inhibiting abnormal cell growth in combination with an anti-tumor agent selected from the group consisting of mitotic inhibitors, alkylating agents, anti-metabolites, intercalating antibiotics, growth factor inhibitors, radiation, cell cycle inhibitors, enzymes, topoisomerase inhibitors, biological response modifiers, antibodies, cytotoxics, anti-hormones, and anti-androgens.

29. The method of claim 28, which comprises administering to said mammal an amount of a compound of claim 1 that is effective in inhibiting abnormal cell growth in combination with a cytotoxic.

30. The method of claim 29, which comprises administering to said mammal an amount of a compound of claim 1 that is effective in treating abnormal cell growth in combination with Taxol®.

31. A method for the inhibition of abnormal cell growth in a mammal which comprises administering to said mammal an amount of the compound of claim 1 that is effective in treating abnormal cell growth in combination with a compound selected from the group consisting of Cyclophosphamide, 5-Fluorouracil, Floxuridine, Gemcitabine, Vinblastine, Vincristine, Daunorubicin, Doxorubicin, Epirubicin, Tamoxifen, Methylprednisolone, Cisplatin, Carboplatin, CPT-11, gemcitabine, paclitaxel, and docetaxel.

32. The method of claim 31, which comprises administering to said mammal an amount of a compound of claim 1 that is effective in inhibiting abnormal cell growth in combination with a compound selected from the group consisting Tamoxifen, Cisplatin, Carboplatin, paclitaxel and docetaxel.

33. A method for treating a mammal having a disease characterized by an overexpression of erbB2, comprising administering to the mammal the compound of claim 1 in an amount that is effective in treating the disease.

34. A method for treating a mammal having cancer characterized by an overexpression of erbB2, comprising administering to the mammal the compound of claim 1 in an amount that is effective in treating said cancer.

35. A method for inducing cell death comprising exposing a cell which overexpresses erbB2 to an effective amount of the compound of claim 1.

36. The method of claim 35, wherein the cell is a cancer cell.

37. The method of claim 36, wherein the cancer cell is a mammalian cancer cell.

38. The method of claim 37, wherein the mammalian cancer cell is a human cancer cell.

39. The method of claim 35, further comprising exposing the cell to a growth inhibitory agent.

40. The method of claim 35, further comprising exposing the cell to a chemotherapeutic agent.

41. The method of claim 35, further comprising exposing the cell to radiation.

42. A method of treating cancer in a human, wherein the cancer expresses the erbB2 receptor, comprising administering to the human a therapeutically effective amount of the compound of claim 1 that has reduced affinity for the erbB1 receptor.

43. The method of claim 42, wherein the cancer is not characterized by overexpression of erbB1 receptor.

44. The method of claim 42, wherein the cancer is characterized by overexpression of the erbB1 and erbB2 receptor.

45. A pharmaceutical composition comprising an amount of a compound according to claim 1 effective to treat a hyperproliferative disorder in a mammal, and a pharmaceutically acceptable carrier.

46. The pharmaceutical composition of claim 45, wherein the composition is adapted for oral administration.

47. The pharmaceutical composition of claim 46, wherein the pharmaceutical composition is in tablet form.

48. A complex formed by contacting E-2-Methoxy-N-(3-{4-[3-methyl-4-(6-methyl-pyridin-3-yloxy)-phenylamino]-quinazolin-6-yl}-allyl)-acetamide with an acid or an reactive equivalent of said acid, wherein said acid is at least one member selected from the group consisting of maleic acid, hydrochloric acid, and phosphoric acid.

49. The complex of claim 48, wherein said acid is maleic acid.

50. The complex of claim 49, wherein said complex is an E-2-Methoxy-N-(3-{4-[3-methyl-4-(6-methyl-pyridin-3-yloxy)-phenylamino]-quinazolin-6-yl}-allyl)-acetamide maleate.

51. The complex of claim 49, wherein said complex is E-2-Methoxy-N-(3-{4-[3-methyl-4-(6-methyl-pyridin-3-yloxy)-phenylamino]-quinazolin-6-yl}-allyl)-acetamide dimaleate.

52. The complex of claim 48, wherein said acid is hydrochloric acid.

53. The complex of claim 52, wherein said complex is an E-2-Methoxy-N-(3-{4-[3-methyl-4-(6-methyl-pyridin-3-yloxy)-phenylamino]-quinazolin-6-yl}-allyl)-acetamide hydrochloride.

54. The complex of claim 53, wherein said hydrochloride is E-2-Methoxy-N-(3-{4-[3-methyl-4-(6-methyl-pyridin-3-yloxy)-phenylamino]-quinazolin-6-yl}-allyl)-acetamide monohydrochloride.

55. The complex of claim 48, wherein said acid is phosphoric acid.

56. The complex of claim 55, wherein said complex is an E-2-Methoxy-N-(3-{4-[3-methyl-4-(6-methyl-pyridin-3-yloxy)-phenylamino]-quinazolin-6-yl}-allyl)-acetamide phosphate.

57. The complex of claim 56, wherein said phosphate is E-2-Methoxy-N-(3-{4-[3-methyl-4-(6-methyl-pyridin-3-yloxy)-phenylamino]-quinazolin-6-yl}-allyl)-acetamide monophosphate.
